(19) United States
(12) Patent Application Publication
(10) Pub. No.: US 2008/0269852 A1
(43) Pub. Date: Oct. 30, 2008

(54) Methods and Apparatus for Thermal Regulation of a Body

(75) Inventors: Charles D. Lennox, Hudson, NH (US); Kevin Allaire, Mattapoisett, MA (US); Jeffrey Radziunas, Wallingford, CT (US); John Thompson, Clinton, CT (US); Paul DiCesare, Easton, MA (US); Greg Hires, Fairfield, CT (US); Gerald Melsky, Lexington, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: MedCool, INC, Wellesley, MA (US)

(21) Appl. No.: 11/910,787

(22) PCT Filed: Apr. 3, 2006
(86) PCT No.: PCT/US06/12561

§ 371 Date: Jun. 16, 2008

Related U.S. Application Data
(60) Provisional application No. 60/669,336, filed on Apr. 7, 2005.

(51) Int. Cl. A61F 7/00

(52) U.S. Cl. 607/104

(57) ABSTRACT

A thermal regulation system includes a thermal exchange collar for application to a neck of a patient, a thermal regulation pad for application to a body region, such as an axilla region of the patient, and a thermal regulation cap for application to a head of the patient.

### CROSS REFERENCE TO RELATED APPLICATIONS

The present application claims the benefit of a U.S. Provisional Application entitled “Methods and Apparatus for Thermal Regulation of a Body,” bearing Ser. No. 60/669,336, filed Apr. 7, 2005, the entire contents of which are hereby incorporated herein by reference.

### BACKGROUND OF THE INVENTION

Patients that suffer from stroke, cardiac arrest, or trauma, such as head trauma, as well as patients that have undergone invasive brain or vascular surgery, are at risk for ischemic injury. Ischemic injury occurs as a result of a lack of oxygen (e.g. lack of oxygenated blood) to an organ, such as caused by a blockage or constriction to a vessel carrying blood to the organ. For example, in the case where a patient suffers a heart attack, typically, a clot can block one of the coronary arteries that carry blood and oxygen to the patient's heart muscle. As a result of the blockage (e.g., an ischemic condition) the patient's heart can experience ischemic tissue injury or heart damage. In the case where a patient suffers from a stroke, typically, a clot blocks the blood supply to a portion of the patient's brain. The blockage, in turn, causes ischemic damage to the brain tissue. For example, as a result of the stroke, the brain experiences a critical or terminal rise in intra-cranial pressure, brain cell death, and a loss of brain function.

Induction of systemic hypothermia (e.g., a hypothermic state) in a patient may minimize ischemic injury when the patient suffers from a stroke, cardiac arrest, heart attack, trauma, or surgery. For example, in the case where the patient suffers a cardiac arrest, the effectiveness of hypothermia is a function of the cooling range (e.g., within a temperature range between approximately 30° C. and 35° C. for example) and duration of the hypothermic state. The effectiveness of the hypothermia is also a function of the amount of time that elapses between the original insult (e.g., cardiac arrest or heart attack) and achievement of protective levels of hypothermia. Also, for trauma and stroke patients, hypothermia aids in controlling swelling of the patient's brain. Furthermore, surgeons typically use hypothermia during brain and other invasive surgeries to protect the brain from surgical interruptions in blood flow.

Systemic hypothermia has historically been applied, such as by immersion of the patient's body in a cool bath, where the depth and duration of hypothermia is limited by the patient's ability to tolerate the therapy. Currently, there are several conventional systemic hypothermia systems available. Such conventional systems include blankets or pads where cooled water is circulated through channels in the walls of the blanket or pad and the patient's body contacts the walls of the blanket.

Attempts have been also made to induce hypothermia in a patient by local cooling the surface of the patient's head. For example, a conventional head-cooling device involves a head cap with a gel substance contained within the walls of the cap. Prior to use, for example, a user (e.g., medical technician) places the head-cooling device in a freezer to reduce the temperature of the gel within the cap. During operation, the user fits the reduced-temperature cap to the head of a patient. The gel within the walls of the cap absorbs heat from the head, thereby cooling the head of the patient.

Other conventional devices induce systemic hypothermia in a patient by providing contact between a tissue region of interest and a cooling fluid. For example, one conventional device includes a flexible hood having multiple ribs or studs disposed on the inner surface of the hood. When a user places the hood on a head of a patient, the ribs or studs contact the head and maintain a fluid circulation space between the head and the hood and an edge, defined by the hood, contacts the patient's skin. A negative pressure source draws a cooling fluid through the flexible hood, under negative pressure, to cause the fluid to contact the scalp of the patient and draw heat away from (e.g., cool) the scalp. Furthermore, application of the negative pressure seals the edges of the hood against the skin of the patient (e.g., a region substantially free of hair).

### SUMMARY OF THE INVENTION

One aspect of the present invention is directed to a method of cooling the heat of a patient to induce hypothermia. At least a portion of the patient's head can be covered with a head cooling device. The device can be configured to form a fluid circulation space bounded by a surface of the device and a surface of the patient's head when the device is placed on the patient's head. Cooling liquid can be circulated through the fluid circulation space while the space is held at negative gage pressure. The liquid can be introduced into the space at a lower level than where the liquid leaves the space. Gas can also be introduced into the fluid circulation space to induce turbulence within the cooling liquid (e.g., the gas can create bubbles in the cooling liquid that induce turbulence). Gas can be introduced through a vent valve, or by allowing air to enter the fluid circulation space by passing air under a head-contacting sealing structure of the head cooling device.

In another aspect, a system for head cooling includes a head cooling device configured to define a fluid circulation space when the device is worn on a patient's head. The fluid circulation space is in fluid communication with a inlet port located at the back of the device, and an outlet port located at the front of the device. The device can further include a gas inlet port for delivering gas into the fluid circulation space. The device can also include a sealing structure configured to contact the patient's head and maintain a negative gage pressure (e.g., between about −0.2 to about −2.0 PSIG) between a turbulent, flowing gas/liquid mixture within the fluid circulation space and ambient atmosphere outside the device. The sealing structure can be configured to allow air to enter the fluid circulation space while hindering liquid from leaking past the sealing structure. At least one pump mechanism can be included with the system, which is in fluid communication with the fluid circulation space and establishes the negative gage pressure. The pump mechanism can be embodied as a first pump mechanism for delivering fluid into the fluid circulation space and a second pump mechanism for removing fluid from the circulation space. The second pump mechanism can be configured to create a higher volumetric flow rate than the first pump mechanism.

A heat exchange collar for removing heat from arteries and veins within a neck of a patient is also described. The collar includes a flexible covering with a fluid circulation space that can communicate with an inlet and an outlet. The flexible covering can effectively exchange heat between a cooling fluid within the fluid circulation space and blood in the arteries and veins. The collar can also include a pressure relief structure coupled to the flexible covering configured to limit pressure on the carotid arteries and jugular veins (e.g., limiting pressure to less than about 0.2 PSIG). The pressure relief structure can include stiffening elements at a first end and a second end of the flexible covering configured to couple together to form an extension extending away from the neck. Such stiffening elements can be biased to promote contact between a heat transfer surface of the flexible covering and a portion of the neck. The pressure relief structure can also include an elastic relief strap configured to be coupled to a first end and a second end of the flexible covering for allowing relative movement between the ends. The structure can also be configured to maintain relatively nominal inter-cranial pressure in the patient.

In place of the pressure relief structure, the flexible covering can have a first end configured to be reversibly attached to a side of the neck and a second end configured to float relative to the first end. In such a configuration, the weight of the flexible covering is capable of maintaining a position of the flexible covering on the neck of a patient.

In another aspect, a thermal regulation system includes a thermal exchange collar for application to a neck of a patient, a thermal regulation pad for application to a body region of the patient, such as an axilla region of the patient, and a thermal regulation cap for application to a head of the patient.

Generally, the thermal exchange collar is configured to provide thermal exchange with the arteries and veins within a neck area. The thermal exchange collar includes a fluid inlet, a fluid outlet, and defines a fluid circulation space between the fluid inlet and the fluid outlet. The thermal exchange collar thermally couples to the neck while minimizing pressure on the patient's airway (to prevent choking of the patient) and while minimizing pressure on the patient's jugular veins and carotid arteries (to allow perfusion of blood carried by the vessels into and out of the patient's head) during operation.

The thermal exchange collar includes a thermal exchange material that contacts the patient, such as a thermally conductive silicone (e.g., silicone impregnated with thermally conductive material, such as metallic material). The thermal exchange collar includes a mesh material within the fluid flow path defined by the collar. The mesh layer helps to limit over inflation of the collar and disturb the fluid boundary layer within the collar during operation. An outer surface of the thermal exchange collar includes an insulation material, such as a foam material, that minimizes heat loss through the outer surface of the collar. The insulation layer also limits the collar from “ballooning” during operation, thereby maximizing contact area between the patient and the collar. The thermal exchange collar is configured as “one size fits all” and allows adjustment of a circumference of the collar, depending upon the neck size of a patient.

In one arrangement, the thermal exchange collar includes stiffening elements that extend away from the patient and that define an opening in the area of the patient's airway to limit choking. The stiffening elements act as springs that force contact between the thermal exchange pads and the carotid arteries within the neck while limiting the amount of pressure exerted by the thermal exchange pad on the patient (e.g., less than approximately 0.2 psi or approximately 10 mmHg).

In one arrangement, the thermal exchange collar has a first end that couples to a flexible strap (e.g., spandex material) secured to the second end of the collar. The flexible strap allows the first end of the collar to move relative to the second end of the collar when the cooling collar inflates due to fluid flowing through the cooling pad. This limits the amount of pressure exerted by the thermal exchange pad on the airway and on the carotid arteries and jugular veins of the patient. Positioning of the flexible strap between the first end and the second end of the collar also limits the ability for a user to generate an “initial tension” on the flexible strap.

In one arrangement, the thermal exchange collar has a first end that is secured to a flexible strap (e.g., spandex material). The flexible strap couples to a second end of the collar. The flexible strap allows the first end of the collar to move relative to the second end of the collar when the cooling collar inflates due to fluid flowing through the cooling pad. This limits the amount of pressure exerted by the thermal exchange pad on the airway and on the carotid arteries and jugular veins of the patient.

In one arrangement, the thermal exchange collar includes a first end having an adhesive tab that attaches to a patient's neck (e.g. to act as an anchor point) and includes a second end having an input and output port. The collar drapes across a patient's neck and the second end of the collar lies against a table or surface carrying the patient. The weight of the collar and the second end of the collar holds the collar in place to maintain adequate thermal contact with the patient while limiting application of pressure to compress the patient's airway or limit perfusion of blood relative to the brain. The second (e.g., free) end of the collar can be clipped to a moveable material adjacent the free end of the collar (e.g., a bed sheet or mattress of a bed) to further secure the collar to the neck area of the patient while allowing displacement or motion of the free end of the collar relative to the surface carrying the patient.

A thermal exchange pad, such as an axilla pad, is configured to provide thermal exchange with a body region, such as an axilla region of a patient. The axilla pad includes a fluid inlet, a fluid outlet, and defines a fluid circulation space between the fluid inlet and the fluid outlet. A thermal exchange surface of the thermal exchange pad thermally couples to the axilla region of the patient. The axilla pad includes connectors that are MRI compatible (e.g., do not interfere with operation of an MRI device when inserted within an MRI device).

The axilla pad includes a strap coupled to the pad and configured to pull the axilla pad into the underarm area of the patient. The strap maintains thermal communication between the axilla region of the patient and the pad.

The axilla pad includes an insulation surface opposing the thermal contact surface. In one arrangement, the insulation surface defines a cut-away area lacking insulation material (e.g., in this configuration the axilla pad cools from both sides of the pad). The cut away area thermally contacts an inner surface of the patient's arm, thereby providing additional thermal exchange with the patient.

An axilla pad system includes an anchoring portion (coupled to a patient in the abdominal area) and an axilla pad defining a first edge and a second edge. The first edge of the axilla pad couples to the anchoring portion using elastic straps. The second edge of the pad couples to the patient using adhesive tabs. The elastic straps, under tension, and the adhesive tabs cause the axilla pad to generate a radial force or load on the patient where the load is directed inward relative to the patient. The radial force increases thermal contact between the patient and the axilla pad, thereby increasing thermal transfer between the patient and the axilla pad.

A thermal exchange (e.g., cooling) cap is configured so that thermal exchange fluid (e.g., cold water) enters at the back (bottom) of the cap and is drained from the front (top) of the cap. This ensures that the circulation space is filled with water during the cooling cycle. At the back of the cap is a second drain port that is activated when the inflow, or cooling cycle is stopped. This drain port provides a means to drain the cap for removal of the cap from the patient's head. A valve can be used to switch between the front drain port and the back drain port. This valve can be manual or automatic. The automatic valve can hydraulically, pneumatically, or electrically actuated.

An additional and important feature is the intentional introduction of air into the circulation space. The air mixes with the water within the circulation space providing agitation to the water. This air agitation greatly increase heat transfer between the cold water and the head surface, and provides for uniform cooling. The water in the circulation space is contained by an elastic seal about the rim of the cap and a negative pressure that is maintained in the circulation space during circulation. The elastic seal is configured to allow air to enter circulation space for agitation purposes, while containing the water within the circulation space. The negative pressure within the circulation space is maintained at a predetermined level by a vent port and a pressure relief valve. When the negative pressure drops below the set point of the pressure relief valve, the valve opens and lets air into the circulation space. The cooling cap also can have one or two intracranial probe ports disposed in the wall of the cap. The probe port provides allows an intracranial probe to be inserted through the wall of the cap and provide a watertight seal around the probe shaft.

A system includes the cap as described above and the console. The console has a source (reservoir) of cold fluid (water) and two pumps. The first pump draws water from the reservoir and delivers it to the cap inlet port under a modest positive pressure. The second pump draws water and air from the circulation space of the cap through one of the drain ports and returns both the water and air to the reservoir. Therefore, the volumetric fluid flow (water and air) through the second pump is greater than the volumetric flow (water) through the first pump. The differential in volumetric flow rates provides the negative pressure in the circulation space.

The cap, in one arrangement, includes a craniotomy protection device. The craniotomy protection device keeps the craniotomy incision dry during head cooling. This craniotomy protection device allows the use of an intracranial probe with a direct water contact type of cooling cap.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 depicts an example of a thermal regulation system.

FIGS. 2A and 2B illustrate an arrangement of a body covering device of FIG. 1.

FIG. 3 illustrates an arrangement of a console of FIG. 1.

FIG. 4 illustrates an example sectional view of the body covering device of FIG. 1.

FIGS. 5A-5C illustrate an arrangement of the body covering device of FIG. 1 as a neck collar.

FIGS. 6A-6B illustrate an arrangement of the body covering device of FIG. 1 as a neck collar.

FIGS. 7A-7C illustrate an arrangement of the body covering device of FIG. 1 as a neck collar.

FIGS. 8A-8B illustrate an arrangement of the body covering device of FIG. 1 as a neck collar.

FIGS. 9A-9B illustrate an arrangement of the body covering device of FIG. 1 as a neck collar.

FIGS. 10-14 illustrate arrangements of the body covering device of FIG. 1 as an axilla pad.

FIGS. 15A-15C illustrate an arrangement of the body covering device of FIG. 1 as an axilla pad.

FIGS. 16-19 illustrate arrangements of the body covering device of FIG. 1 as an axilla pad.

FIGS. 20-30 illustrate arrangements of the head covering device of FIG. 1.

FIG. 31 illustrates a resuscitation system that includes a thermal regulation system, such as illustrated in FIG. 1.

### DETAILED DESCRIPTION

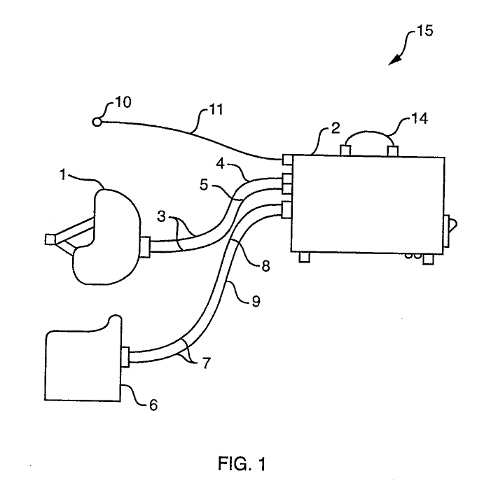

FIG. 1 depicts an arrangement of a thermal regulation system 15. The thermal regulation system 15 includes a head covering device 1, a console 2, a body covering device 6, and a body temperature sensor 10. The thermal regulation devices and systems described in U.S. application Ser. No. 10/424,391 entitled “Method and Device for Rapidly Inducing Hypothermia” and U.S. application Ser. No. 10/706,327 entitled “Method and Device for Rapidly Introducing and then Maintaining Hypothermia”, herein incorporated by reference.

The head covering device 1, in one arrangement, is removably connected to console 2 by umbilical 3 having, for example, a fluid inlet tube 4 and a fluid outlet tube 5. The head covering device 1 operates to adjust the body temperature, such as a localized body temperature of a patient. The body covering device 6 is removably connected to console 2 by umbilical 7 having, for example, a fluid inlet tube 8 and a fluid outlet tube 9. The body covering device 6 operates to adjust the body temperature, such as a localized body temperature of a patient.

The body temperature sensor 10, in one arrangement, is removably connected to console 2 by a body temperature sensor lead 11. The body temperature sensor 10 is configured to attach onto (e.g., on an outer surface) or within (e.g., within a natural orifice) a patient's body to measure the temperature of the patient during operation of the thermal regulation system 15. In one arrangement, the body temperature sensor 10 is an esophageal temperature sensor configured to insert within an esophagus of a patient to measure core body temperature. In another arrangement, the body temperature sensor in a bladder temperature sensor or a tympanic temperature sensor configured to insert within a bladder or ear, respectively, of a patient.

The console 2 contains a thermal regulation fluid, such as cooling fluid held within a reservoir and provides the thermal regulation fluid to the head-cooling device 1 and the body-cooling device 6 under positive gage pressure (e.g., from a pressure source or positive gage pressure source, such as a water pump, associated with the console 2). The console 2 also has, in one arrangement, a thermal adjustment device. For example, the thermal adjustment device includes a fluid cooling mechanism for cooling the thermal regulation fluid. The console 2 also includes a flow rate adjustment mechanism to adjust the flow of thermal regulation fluid from console 2 to the head covering device 1 and the body covering device 6, according to signals received from the body temperature sensor 10, during operation. As such, for example, the console 2 controls body cooling by controlling the delivery of cooling fluid to the patient during operation of the cooling system 15 and the duration of application of the cooling fluid. In one arrangement, the console 2 has a handle 14 that allows a user to grasp and transport the console 2 to a patient.

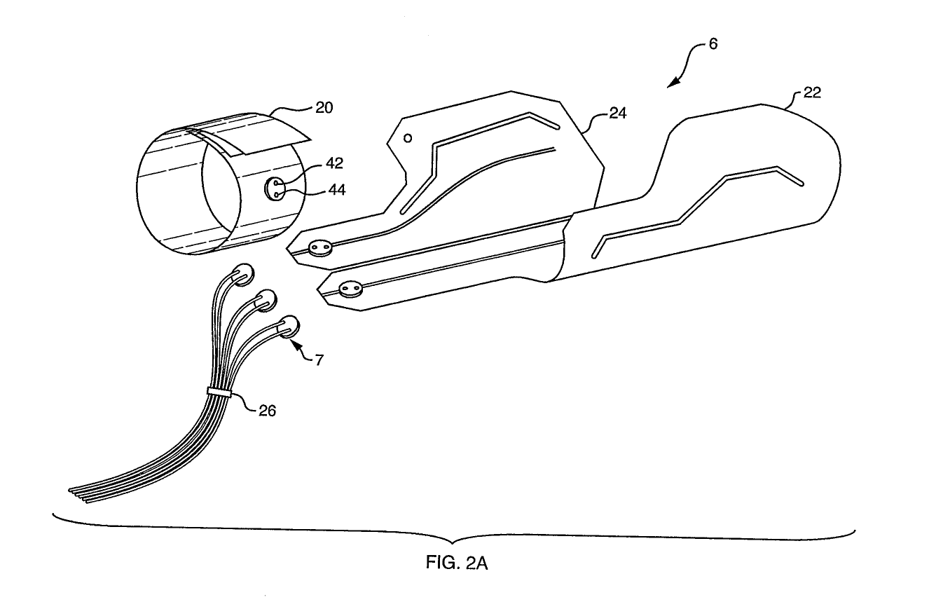

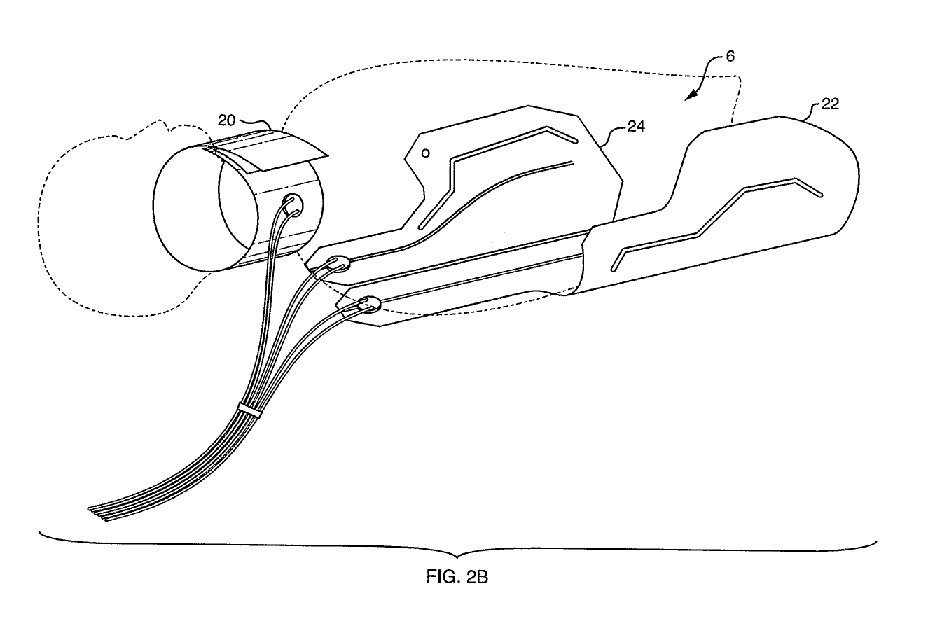

FIGS. 2A and 2B illustrate various arrangements of the body covering device 6 shown in FIG. 1. The body covering device 6 can be configured as a collar 20 (e.g., a neck collar), an axilla pad 22, or a back pad 24. The collar 20, axilla pad 22, and back pad 24 attach to the console 2 via the umbilical 7. The umbilical 7 include a strain relief portion 26 that allows the umbilical 7 to stretch relative to the body covering devices 6 and the console 2, thereby minimizing accidental detachment of the umbilical 7 from either the devices 6 or the console 2 during operation, when exposed to a tensile force.

Typically during operation, a user utilizes the collar 20 and the axilla pad 22 to adjust the temperature (e.g., core temperature) of the patient. The user can utilize the back pad 24 if the patient core temperature is non-responsive or slow to respond.

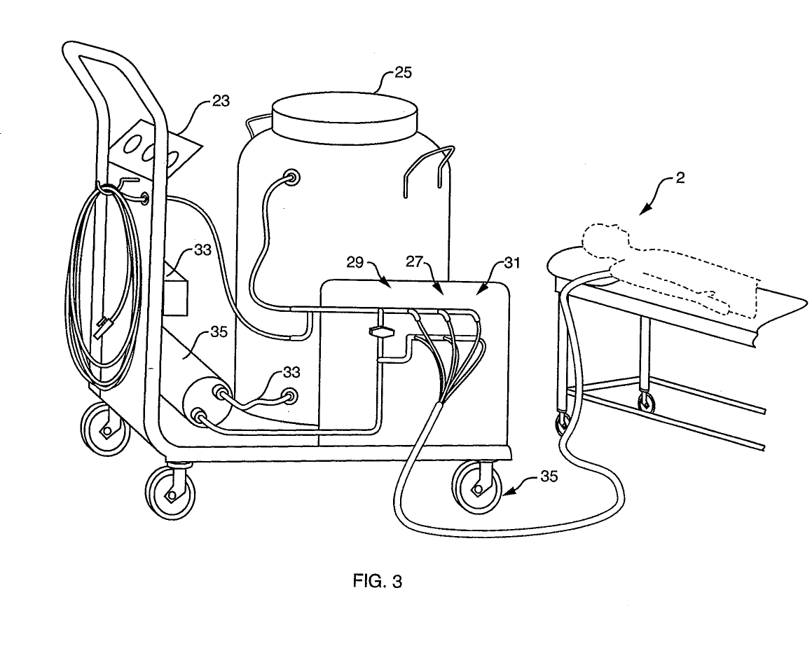

In one arrangement each of the collar 20, axilla pad 22, and back pad 24 couple to a console 2 as illustrated in FIG. 3. During operation, the user or clinician controls three variables associated with the console 2: an ON/OFF power supply 23, an amount of ice contained in a reservoir 25, and a number of accessories attached to the system, including a throttling valve position 27. The need to replace ice in the reservoir 25 is indicated by a temperature reading on a display (e.g., an LCD) on the pump cart or console 2. The clinician adjusts the throttling valve 27 to a clearly marked position 29 indicating the number of accessories or body covering devices 6 attached to the console 2. Additional features of the console include a manifold tubing set 31 with inlet and outlet for each accessory, a bypass loop with throttling valve, a drain port 33, 120 VAC plug 50-60 Hz 33, a cart with caster 35, and a pump with AC/DC transformer 37. In one arrangement, the console 2 includes a bypass release valve (not shown). If a pressure within one or more of the body covering devices 6 rises above a threshold value (e.g., 2 psi) the bypass release valve opens and directs thermal fluid circulating within the devices 6 into the reservoir 25. The release valve limits over inflation and potential damage to the body covering devices or pads 6.

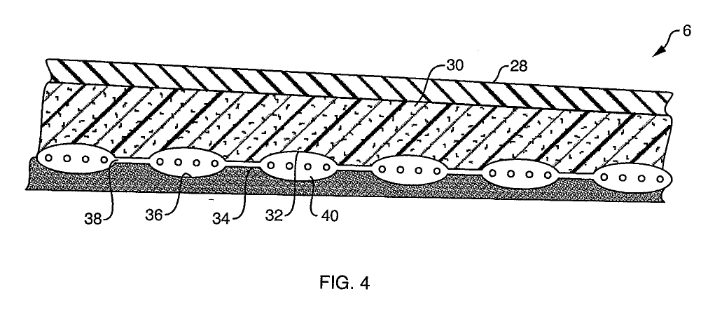

Each of the collar 20, axilla pad 22, and back pad 24 are formed of multiple layers as illustrated in FIG. 4. For example, each body covering device 6 includes an outer layer 28, such as formed of a neoprene material, a foam layer 30, an outer flow channel layer 32, a mesh layer 34, an inner flow channel layer 36, and a silicone inner layer 38.

The outer flow channel layer 32 and the inner flow channel layer 34, such as formed from a vinyl or urethane material, define fluid flow channels 40 within the body covering device 6. The fluid flow channels 40 carry fluid (e.g., thermal exchange fluid or cooling fluid) from the console 2, through the device 6, and back to the console 2. The channels 40 defined by the inner layer 34 and the outer layer 32 include the mesh layer 34. The mesh layer 34, such as a fabric type material, helps to limit over inflation of the body covering device 6. Additionally, as fluid flows through the channels 40, the mesh material 34 creates turbulence of the fluid within the channels 40 and minimizes fluid stagnation or boundary layer effects within the channels 40. The foam layer 30 helps to insulate the channels 40 against thermal conductivity with the atmosphere. For example, the foam layer 30 maintains the temperature of a cooling fluid circulating through the channels 40 at a substantially constant temperature and minimizes heat exchange between the cooling fluid within the body covering device or pad 6 and the atmosphere. The silicone layer 38 thermally contacts the skin of a patient during operation. In one arrangement the silicone layer 38 includes, or is impregnated with, a thermally conductive material, such as a metal material. For example, the metal material includes copper shavings or steel shavings. The metal materials included within the silicone layer 38 increase thermal transfer between the skin of the patient and the fluid carried by the channels 40 within the pad 6.

Returning to FIG. 2A, the body covering devise 6, in one arrangement, is configured as a collar 20. Generally, the collar 20 provides thermal exchange with the arteries and veins within neck area of a patient. The collar 20 includes a fluid inlet 42, a fluid outlet 44, and a fluid circulation space between the fluid inlet 42 and the fluid outlet 44 (e.g., the flow channels 40 as illustrated in FIG. 4). The relative size of the collar 20 is adjustable by a user, relative to a patient's neck (e.g., the collar 20 is configured as “one-size-fits-all”). The user adjusts the size of the collar 20 depending on the neck size of the patient to ensure thermal contact between the collar 20 and the neck of the patient.

Embodiments of the collar 20 minimize the amount of pressure placed on a patient's airway when the collar 20 is placed on the neck of the patient. The collar 20, thereby, minimizes or prevents choking of the patient. Also when the collar is contact with the neck of the patient, embodiments of the collar minimize a pressure placed on the patient's jugular veins and carotid arteries. As such, the collar 20 minimally limits perfusion of blood, as carried by the arteries and veins, into and out of the patient's head. In such embodiments, the collar 20 places a pressure on the neck less than a pressure of approximately 0.2 pounds per square inch and maintains relatively normal inter-cranial pressure within the patient during use.

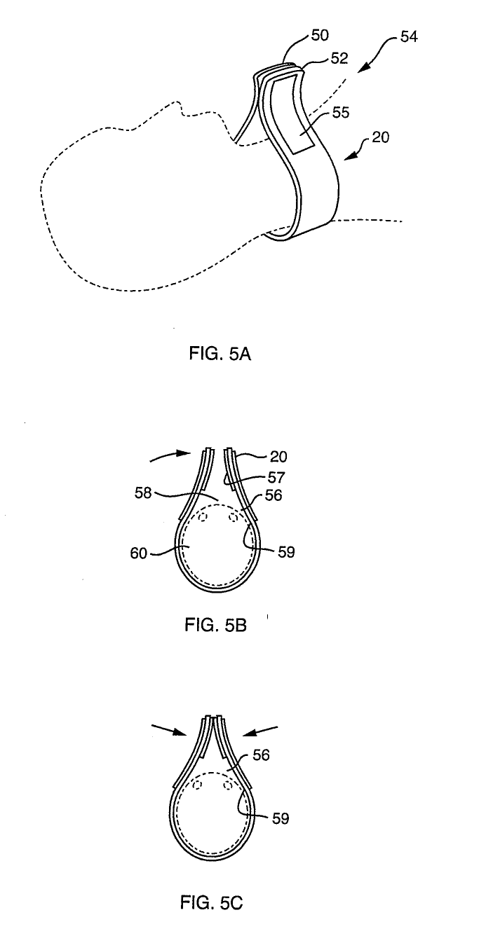

FIGS. 5A-5C illustrate an arrangement of the collar 20. The collar 20 has a first end 50 and a second end 52 where the first end 50 and the second end 52 define an extension portion 54 or “duck bill” that extends away from an airway area 56 of the patient. The first end 50 and the second end 52 of the collar 20 attach together using an attachment mechanism 57, such as a VELCRO hook and loop material. The extension portion 54 is formed of semi-flexible, plastic stiffening elements 55 that maintains the first end and the second end 54 of the collar away from the airway 56 of the patient to define a space 58 between the collar 20 and the neck of the patient (e.g., the airway 56). By maintaining the space 58 between the collar 20 and the patient, the collar 20 prevents or limits choking of the patient or choking or compression on the airway 56 of the patient. Additionally, the stiffening elements 55 act as leaf springs that force contact between a thermal transfer portion 59 of the collar 20 and the carotid arteries 60 of the patient. Such thermal contact allows thermal adjustment (e.g., cooling or heating) of the blood carried by the carotid arteries during operation.

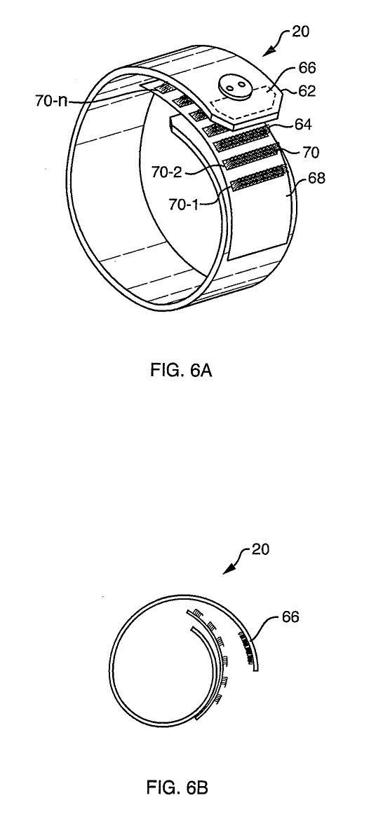

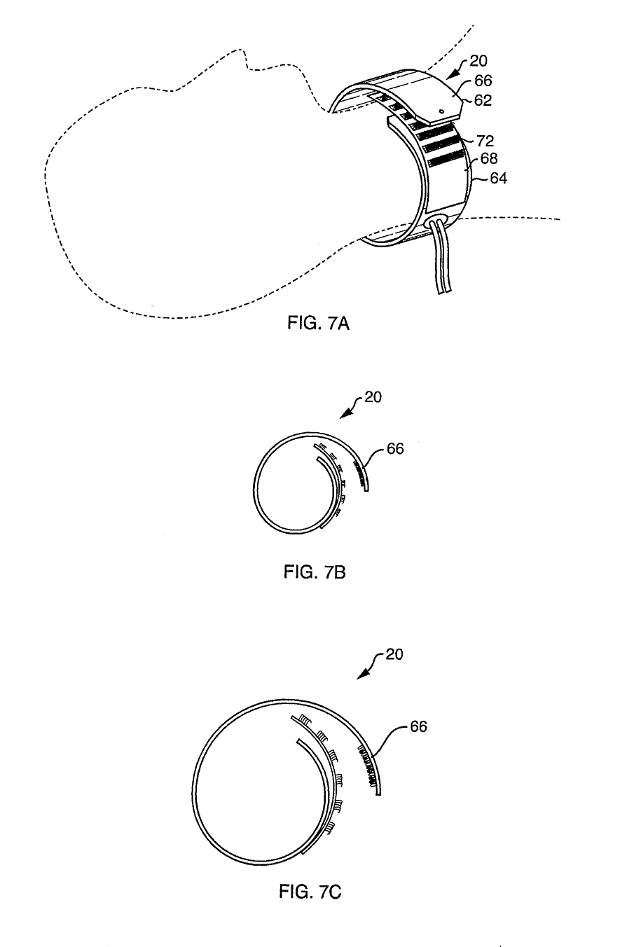

FIGS. 6A-6B and FIGS. 7A-7C illustrate an arrangement of the collar 20 where the collar 20 includes an elastic strain relief strap 68. For example, the collar 20 includes a first end 62 and a second end 64. The first end 62 of the collar 20 includes a VELCRO loop pile material 66. The second end 64 of the collar 20 includes an elastic or flexible strap 68. For example, the elastic strap 68 is formed of a spandex material. The elastic strap 68 secures or couples to the second end 64 of the collar 20. The elastic strap 68 includes a VELCRO hook material 70. For example, as shown in FIG. 6A the hook material 70 is configured as separate hook material elements 70-1, 70-2, 70-N on the elastic band 68. In another example, as shown in FIG. 7A, the loop material is configured as a single hook material pad 72.

During operation, a user attaches the first end 62 of the collar to the elastic band 68 of the second end 64 of the collar 20. The flexible strap 68 allows the first end 62 of the collar 20 to move relative to the second end 64 of the collar 20 when the collar 20 inflates (e.g., as caused by fluid flowing through the channels 40 of the collar 20). As the collar 20 inflates, the elastic strap 68 stretches to allow separation of the first end 62 of the collar 20 relative to the second end 64 of the collar 20. As such, the elastic strap 68 limits the amount of pressure exerted by the collar 20 on both the airway and the carotid arteries and jugular veins of the patient. As such, the elastic strap 68 of the collar 20 minimizes choking of the patient and maintains the inter-cranial pressure within the patient's head at a relatively normal or constant amount. Additionally, as the user couples the first end 62 of the collar 20 to the second end 64 of the collar 20 using the elastic strap 68 (e.g., by coupling the loop pile material 66 of the first end 62 of the collar 20 to the hook material 70 of the elastic strap 68, the user is unable to generate an initial tension or preload on the elastic strap 68. Because the user cannot apply a preload or pre-stretch to the elastic strap 68, thereby changing the spring characteristics of the elastic strap 68, the configuration of the collar limits the user from over tightening the collar. The collar configuration, as shown in FIGS. 6A-6B and FIGS. 7A-7C, ensures that the patient is not compromised by an improper application of the collar 20.

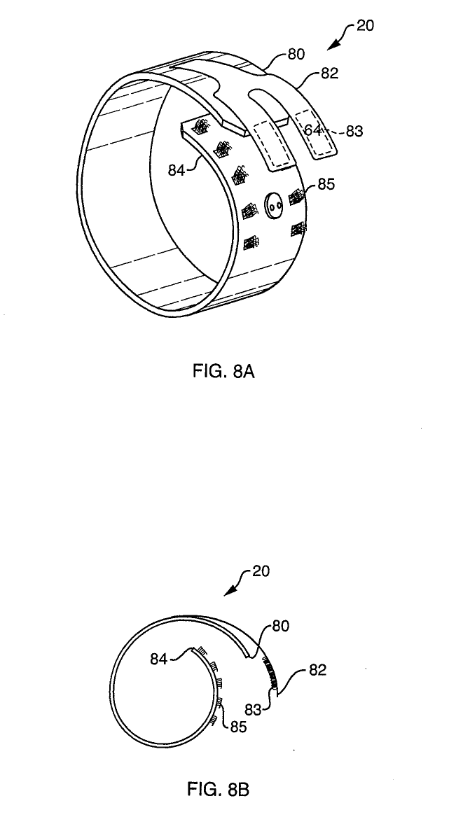

FIGS. 8A and 8B illustrate an arrangement of the collar 20. The thermal exchange collar 20 has a first end 80 secured to a flexible or elastic strap 82 (e.g., such as a spandex material). The flexible strap 82 includes a VELCRO type loop pile material 83 that couples to a second end 84 of the collar 20 having a hook material 85. When a user applies the collar 20 to a patient, the user can apply a preload or tension in the flexible strap 82 prior to securing the flexible strap to the second end 84 of the collar 20. The flexible strap 82 allows the first end 80 of the collar 20 to move relative to the second end 84 of the collar 20 when the collar 20 inflates (e.g., due to fluid flowing through the channels 40 of the collar 20). The flexible strap 82 limits the amount of pressure exerted by the collar 20 on the airway and on the carotid arteries and jugular veins of the patient during operation of the collar 20.

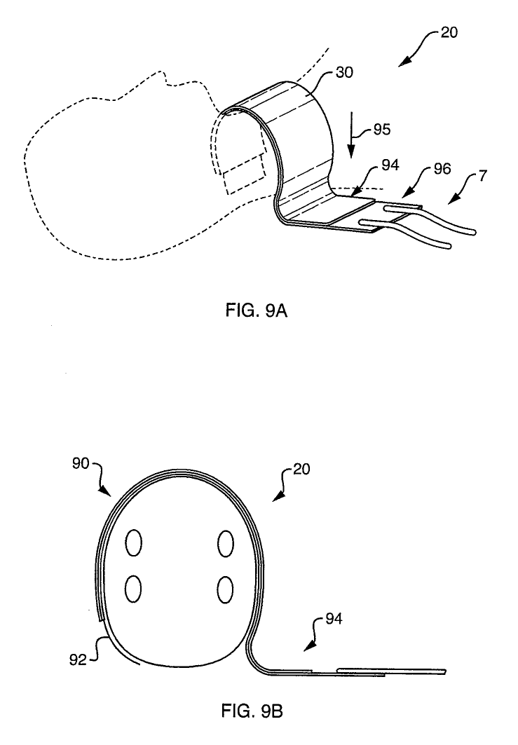

FIGS. 9A and 9B illustrate an arrangement of the collar 20 where the weight of the collar 20 and geometric configuration of the collar 20 help to maintain thermal communication between the collar 20 and the neck of the patient. The collar 20 includes a first end 90 having an adhesive tab 92. The collar 20 also includes a second end 94 that includes input and output connections 96, for connection to the umbilical 7, to attach the collar 20 to the console 2.

During operation, a user couples the adhesive tab 92 (e.g., a tab having a medical grade pressure sensitive adhesive) to one side of the patient's neck and drapes the collar 20 across or about the patient's neck. The user places the second end 94 of the collar 20 against a table or gurney upon which the patient lays. The adhesive portion 92 secures the first end 90 of the collar 20 to the patient. The weight of the collar (e.g., the weight of the foam pad 30 on the outside surface of the collar 20) and the weight of the hoses 96 attaches to the second end 94 of the collar 20 help to generate a force 95 on the neck of the patient. The weight maintains the position of the collar 20 on the patient and maintains thermal communication between the collar 20 and the neck of the patient. With the collar 20 having a free end (e.g., the second end 94 that rests on the table), during operation, as the collar 20 expands (e.g., as caused by flow of fluid through the channels 40 of the collar 20), the free end 94 of the collar 20 moves relative to the table, thereby minimizing exertion of excessive pressure on the airway of the patient or on the carotid arteries and jugular veins of the patient. Because the collar 20 essentially “free floats” with the adhesive tab 92 acting as the anchor point, the collar 20 minimizes choking of the patient and allows adequate perfusion of blood into and out of the patient's head during operation.

In one arrangement, the free end 94 of the collar 20 secures to a moveable material associated with the table. For example, assume the table includes a sheet covering the table where the sheet can move relative to the table. A user can fasten the free end 94 of the collar 20 to the sheet, such as by using a safety pin. Such fastening helps to secure the free end 94 of the collar 20. Additionally, because the sheet can move relative to the table, as fluid travels within the collar 20 to inflate the collar 20, the free end 94 of the collar 20 and the sheet move as a unit to minimize choking of the patient and allows adequate perfusion of blood into and out of the patient's head during operation.

Returning to FIG. 2A, the body covering device 6, in one arrangement, is configured as a thermal exchange pad or axilla pad 22 that thermally couples with an axilla region of a patient. The thermal exchange pad or axilla pad 22, in such an arrangement, enhances thermal adjustment of a patient when used in conjunction with the head covering device 1 as shown in FIG. 1. For example in the case where the head covering devise 1 delivers and circulates cooling fluid to the scalp of a patient, the axilla pad 22 also delivers cooling fluid to the axilla region of the patient to reduce the temperature of the core body temperature of the patient, such as to a hypothermic level.

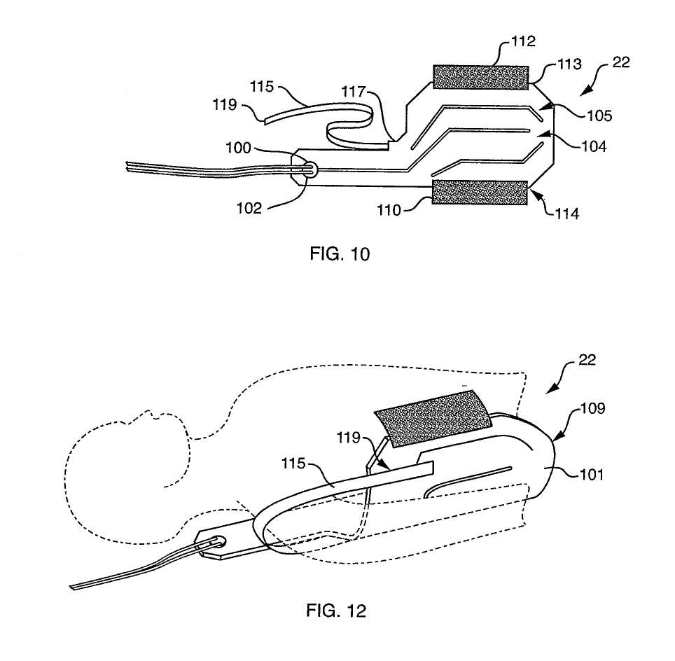

FIG. 10 illustrates an example of the axilla pad 22. The axilla pad 22 includes a fluid inlet 100, a fluid outlet 102, and a fluid circulation area 104 defined between the fluid inlet 100 and the fluid outlet 102. The fluid inlet 100, a fluid outlet 102 (e.g., tubing attachment points) orient within a close proximity relative to each other to improve ease of use. As shown in FIG. 10, the fluid circulation area 104 defines multiple channels 105 that carry the fluid from the inlet 100 to the outlet 102. The axilla pad 22 includes an outer or insulation layer 107 and an inner or thermally conductive layer 109 (e.g., the such as the silicone layer 36 illustrated in FIG. 4). During operation, the fluid flow channels 105 of the axilla pad 22 carry the thermal exchange fluid throughout the axilla pad 22 to create thermal contact between the fluid and the thermal conductive layer 109 of the axilla pad 22. As such, the axilla pad 22 provides thermal exchange with an axilla region of a patient.

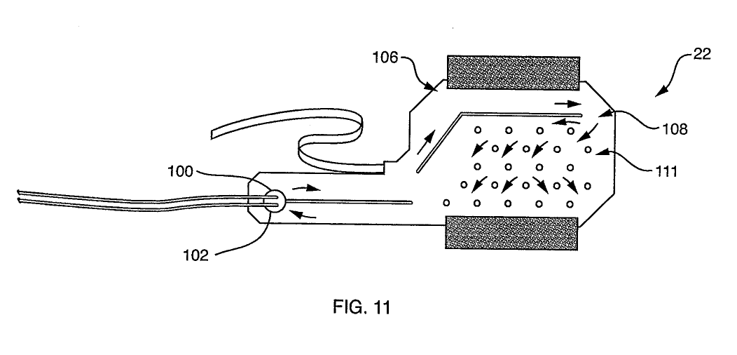

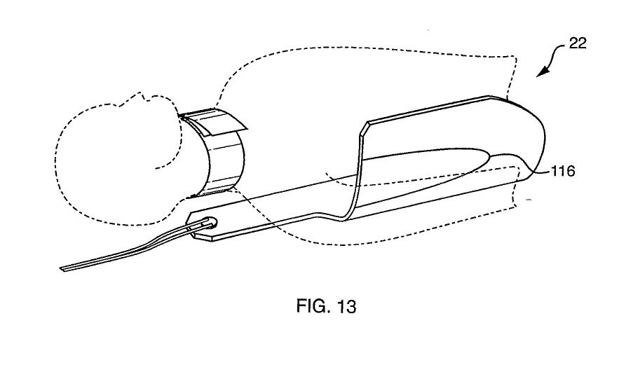

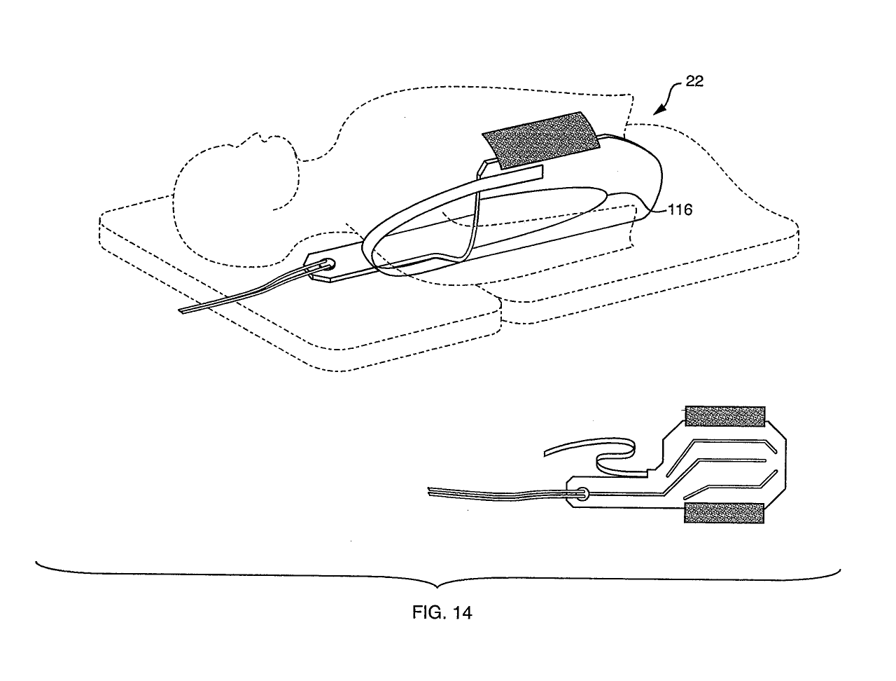

FIG. 11 illustrates an alternate arrangement of the fluid circulation area 104 of the axilla pad 22. As illustrated, the fluid circulation area 104 of the axilla pad 22 includes a fluid inlet channel 106 in a plurality of extensions or protrusions 108 within an outlet flow path 111 of the axilla pad 22. During operation, fluid flows from the inlet 100 through the fluid inlet channel 106. As the fluid exits the fluid inlet channel 106, the fluid contacts the protrusions or dimples in the outlet flow path 111. The dimples 108 provide an even distribution of fluid flow within the axilla pad from the inlet 100 to the fluid outlet 102. This minimizes the dominance of certain flow channels over others within the axilla pad 22 and maintains adequate thermal exchange between the fluid and the axilla region of the patient.

As illustrated in FIGS. 10 and 12, the axilla pad 22 includes adhesive portions 110, 112 oriented on the rear edge 114 and top edge 113, respectively of the axilla pad 22. For example the axilla pad 22 includes a rear adhesive portion 110 that secures the rear edge 114 of the axilla pad 22 to the back or lumbar area of the patient. The axilla pad 22 also includes an abdominal adhesive portion 112 that attaches the top edge 113 to an abdominal area of the patient. Both adhesive patches 110 and 112 work in conjunction with each other to conform the thermal exchange surface 109 of the axilla pad 22 to an axilla region (e.g., under arm region) of the patient. As such, the adhesives 110, 112 maximize thermal transfer between the axilla pad 22 and the axilla region of the patient.

Also as illustrated in FIGS. 10 and 12 the axilla pad 22 includes a strap 115 having a first end 117 secured to the pad 22 and having a free second end 119 that removably attaches to the pad 22 via a VELCRO type attachment (e.g., hook and loop material configuration). During operation, a user loops the strap 114 about a shoulder of the patient and secures the second end 119 of the strap 15 to the axilla pad 22. As such, the strap 115 forces the axilla pad 22 into the underarm region of the patient. The strap 115 ensures proper positioning of the pad 22 relative to the axilla region of the patient and therefore ensures optimal thermal contact between the axilla pad 22 and the axilla region of the patient. The configuration of the strap 115 also limits interference of the axilla pad 22 with a pressure cuff applied to the arm of the patient.

FIGS. 13 and 14 illustrate another arrangement of the axilla pad 22. The axilla pad 22 includes an outer insulation layer 109 that helps to maintain the temperature of the thermal exchange fluid within the axilla pad 22 during operation. As illustrated in FIGS. 13 and 14, the axilla pad 22 includes a “cutaway region” 116 in the insulation layer 109. The “cutaway area” 116 is defined as a section of the axilla pad 22 where the insulation layer has been removed. As such, the “cutaway area” 116 exposes a surface of the thermally conductive layer 109 through the insulation layer 109 of the pad 109. For example as shown in FIGS. 13 and 14, the “cutaway region” 116 of the axilla pad 22 exposes a thermal transfer surface relative to an inner arm surface of the patient.

During operation, the axilla pad 22 provides thermal exchange between the thermal exchange surface of the pad and the axilla region of the patient, as indicated above. The axilla pad 22 with the “cutaway region” 116 also provides thermal exchange with the inner arm of the patient. The thermal exchange pad 22 provides enhanced thermal exchange with the patient because the pad 22 contacts not only the axilla region of the patient but the inner arm region of the patient as well. As such the thermal exchange pad or axilla pad 22 allows cooling to occur from both sides of the pad 22 thus, enabling thermal exchange (e.g., cooling) of the major vessels in the axilla and inner arm region.

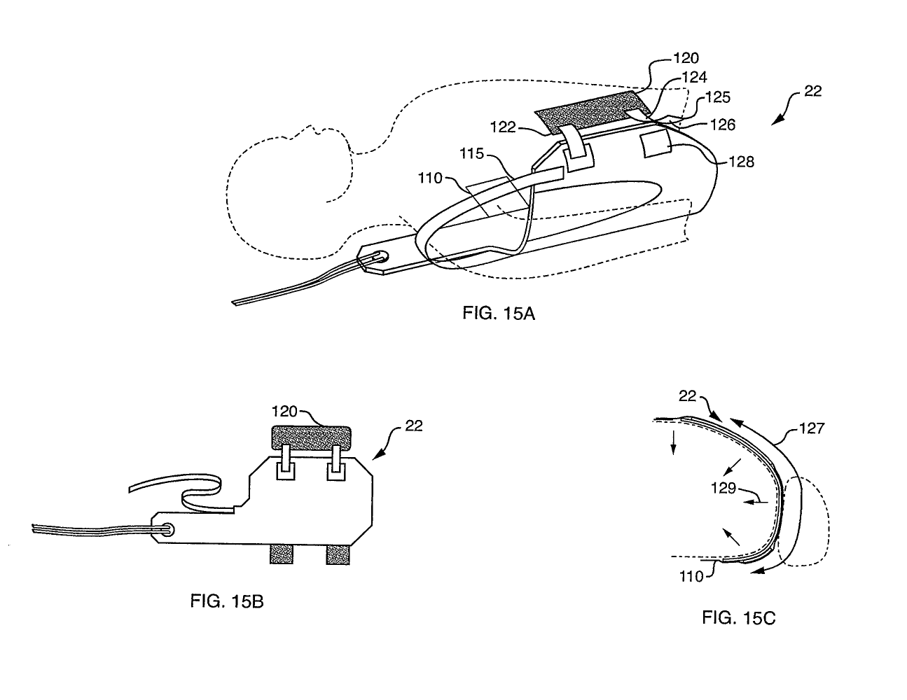

FIGS. 15A through 15C illustrate an arrangement of the axilla pad 22 where the axilla pad 22 has a separable anchoring portion or pad 120 that secures the axilla pad 22 to the patient during operation. The anchor pad 120 includes a pressure sensitive adhesive 122 and elastic fastening straps 124. The elastic fastening straps 124 include free ends 125 having a hook type material for fastening to a loop pile material associated with the axilla pad 22.

During operation a user adheres the pressure sensitive adhesive of the anchor pad 120 to abdominal or chest region of the patient. The user applies the axilla pad 22 to the patient. For example, the user adheres the adhesive strip 110 located along the back portion 114 of the axilla pad 22 to the back of the patient. The user places the axilla pad 22 against the axilla region of the patient and attaches the strap 115 to the axilla pad 22 to force the axilla pad 22 into the axilla region of the patient. The user then stretches (e.g., preloads) the straps 124 of the anchor 120 and attaches the hook material 126 of the free ends 125 to the loop pile material 128 located on the insulation layer 107 of the axilla pad 22. By applying a tension to the straps 124 and fastening the free ends 125 of the straps 124 to the axilla pad 22 the anchor portion 120, in combination with the adhesive tab 110 attached to the back surface of the patient, creates a tension on the axilla pad 22. As shown in FIG. 15C, the tension generated on the pad 22 by the anchoring portion 120 and the adhesive portion 110 creates a substantially consistent radial force 127 within the pad 22 thereby causing the pad 22 to generate a substantially consistent inward force 129 on the patient. The radial force 127 and inward force 129 increases thermal contact between the patient and the axilla pad 22 thereby increasing thermal transfer between the patient and the axilla pad 22.

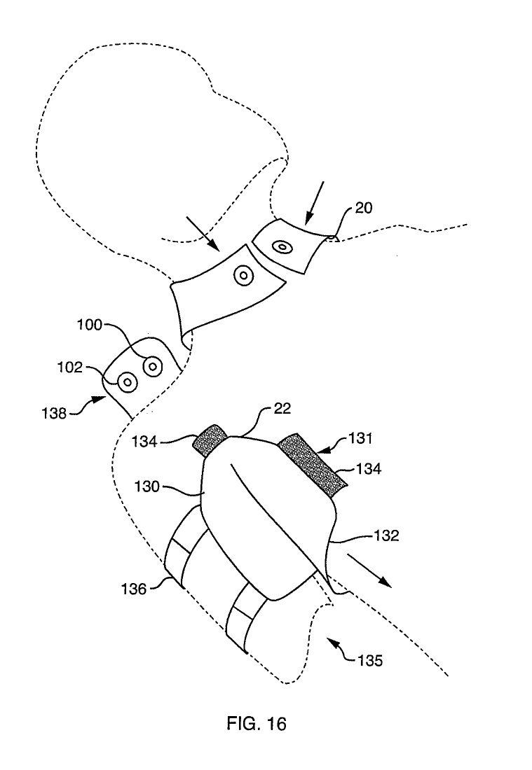

FIGS. 16 through 19 illustrate other arrangements of the axilla pad 22. For example as illustrated in FIGS. 16 through 19, the axilla pad 22 is configured as a U-shaped pad 131 having an outer insulation surface 130 and an inner thermal transfer surface 132. The thermal transfer surface 132 extends through the axilla region of the patient and to the inner arm region 135 of the patient. The use of the U-shaped pad 131 maximizes thermal transfer between the pad 131 and the patient because of the thermal contact between the pad 131 and both the axilla region and the pad and the inner arm region 135 of the patient.

The U-shaped pad 131 includes adhesive portions 134 that attach to the patient's chest and shoulder area to secure the U-shaped pad 131 to the patient. In the arrangement of the U-shaped pad 131 the axilla pad also includes bands 136, such as elastic bands having VELCRO type hook and loop pile attachment elements that secure the U-shaped pad 131 or a portion of the U-shaped pad 131 to the arm of the patient.

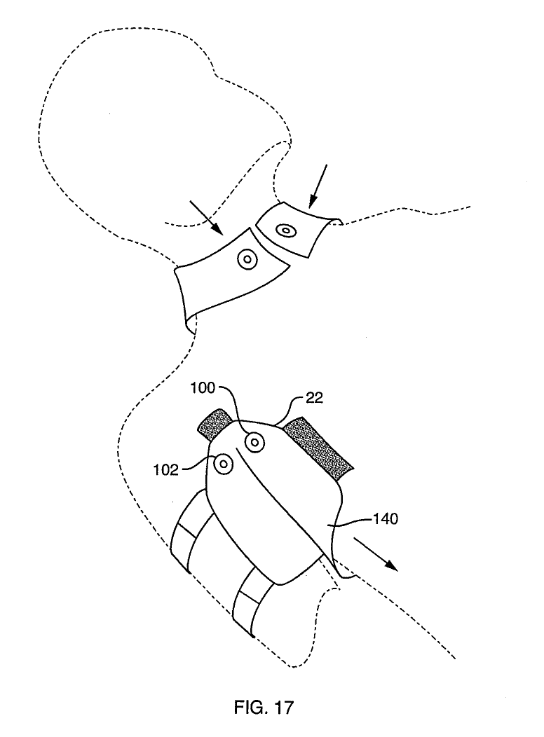

FIG. 16 shows the U-shaped pad 13 having a fluid inlet (e.g., inlet port) 100 and a fluid outlet (e.g., outlet port) 102 oriented in proximity to a collar 20 of a patient. The inlet 100 and outlet 102 form part of an extension portion 138 of the U-shaped pad 131. In such a configuration, the fluid inlet 100 and outlet 102 orient away from the arm of the patient and limit interference of an umbilical 7, attached to the fluid inlet 100 and outlet 102, with a pressure cuff applied to the arm of the patient. FIG. 17 illustrates the U-shaped pad 131 having a fluid inlet 100 and outlet 102 oriented along an upper surface of the U-shaped pad 131 (e.g., a thermal insulation surface of the U-shaped pad 131). Such an orientation also limits interference of an umbilical 7, attached to the fluid inlet 100 and outlet 102, with a pressure cuff applied to the arm of the patient.

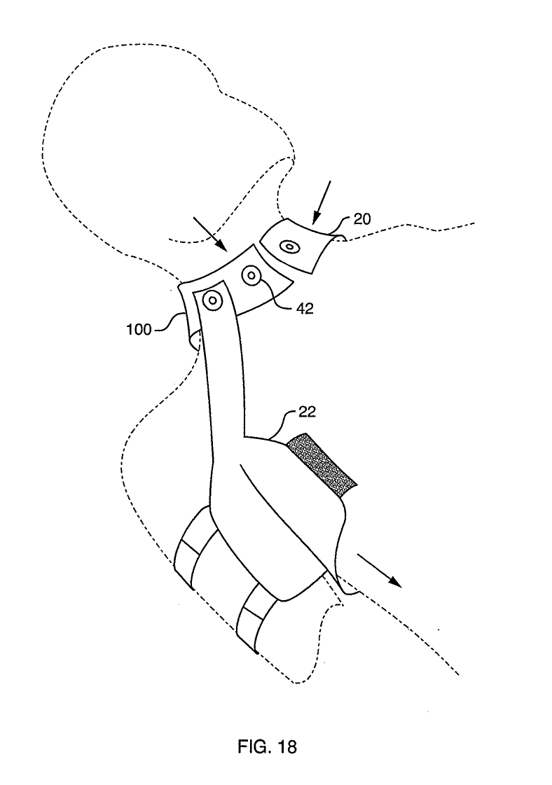

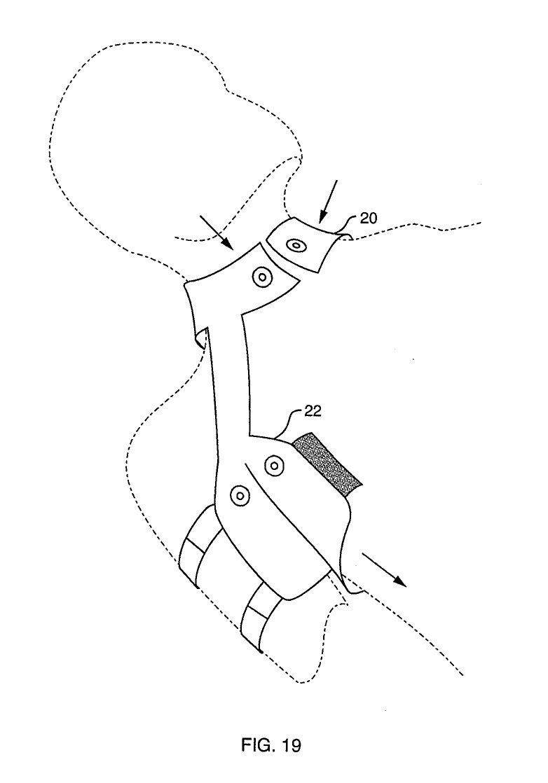

FIG. 18 illustrates the axilla pad having a fluid inlet 100 that couples to a fluid inlet port 42 of the collar 20. In such a configuration, both the U-shaped pad 131 and the collar 20 attach to a control console 2 by a single umbilical connector 7. FIG. 19 illustrates an arrangement of the axilla pad 22 where the axilla pad 22 is integrally formed with the collar 20.

Returning to FIG. 1, the thermal regulation system 15 includes a head covering device 1. The head covering device 1 and the body covering device 6 act in conjunction with each other to adjust a body core temperature of a patient. For example the head covering device 1 and the body covering device 6 operate to lower the body core temperature of the patient to induce a hypothermic state in the patient.

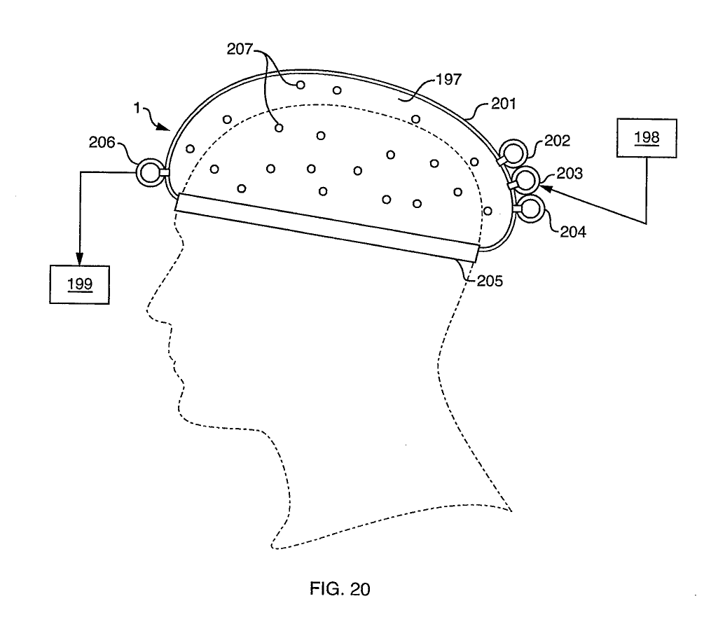

FIG. 20 illustrates an arrangement of the head covering device 1 or cooling cap. The cooling cap 1 includes a covering portion (e.g., a compliant elastomeric membrane or a hard shell polycarbonate material) 201 having a sealing element 205 (e.g., a sealing membrane) disposed about a perimeter of the covering portion 201 and defines a fluid circulation space 197 with a patient's head. The cooling cap 1 includes a fluid inlet 203 that couples to a first pump 198 and delivers thermal exchange fluid to the fluid circulation space 197 at a first flow rate. The cooling cap 1 also includes a fluid outlet 206 that couples to a second pump 199 and removes fluid from the fluid circulation space 197 at a second flow rate, where the second flow rate is greater than the first flow rate. The difference in flow rates between the first pump 198 and the second pump 199 carries fluid through the fluid circulation space 197 at a relatively high flow rate (e.g., between approximately 3 liters/min and 6 liters/min), thereby providing thermal exchange between the patient's head and the thermal exchange fluid. For example, the second flow rate can exceed the first flow rate by an amount in the range of about 25% to about 1000% of the first flow rate. Also, the difference in flow rates between the first pump 198 and the second pump 199 creates a slightly negative pressure within the fluid circulation space 197 relative to the atmosphere outside the head covering device 1. The negative pressure in combination with the sealing element 205 maintains the thermal exchange fluid substantially within the fluid circulation space 197 and minimizes leakage of the fluid past the perimeter of the cap 1. In one example, the negative pressure difference can be in the range of about 0.2 PSIG to about 2.0 PSIG.

In one arrangement, the cap includes a vent port or vent valve 202. The vent valve 202 provides air into the fluid circulation space 197 to maintain a slightly negative pressure within fluid circulation space 197 (e.g., as caused by the out flow from the fluid outlet being greater that the inflow from the fluid inlet). Additionally, the air creates turbulence within the fluid circulation space 197 defined by the cooling cap 1, thereby minimizing stagnation of fluid flow or boundary layer effects relative to the inner wall of the covering portion 201 of the cap 1.

In one arrangement, the sealing element 205 allows air to enter the fluid circulation space 197 from the atmosphere (e.g., the negative pressure within the fluid circulation space 197 draws air past the sealing element 205 and into the circulation space 197) to maintain a slightly negative pressure within fluid circulation space 197 (e.g., as caused by the out flow from the fluid outlet 206 being greater that the inflow from the fluid inlet 203). Additionally, the air creates turbulence within the fluid circulation space 197 of the cooling cap 1, thereby minimizing boundary layer effects relative to the inner wall of the covering portion 201 of the cap 1.

In one arrangement, the inlet 203 orients at the back of the cap 1 (e.g., base of skull region) and the fluid outlet 206 orients at the top of the cap 1 (e.g., forehead region). Such a design allows any air introduced into fluid circulation space 197 to flow (e.g., float) toward the top of the cap 1, to the outlet 206, for removal from the cap 1 by the pump 199. The relative positioning of the inlet 203 and the outlet 206, therefore, minimizes the creation of air pockets within the fluid circulation space 197 that can decrease the cooling efficiency or thermal transfer between the patient's scalp and the thermal exchange fluid.

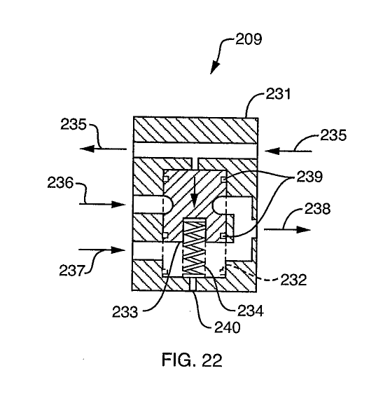

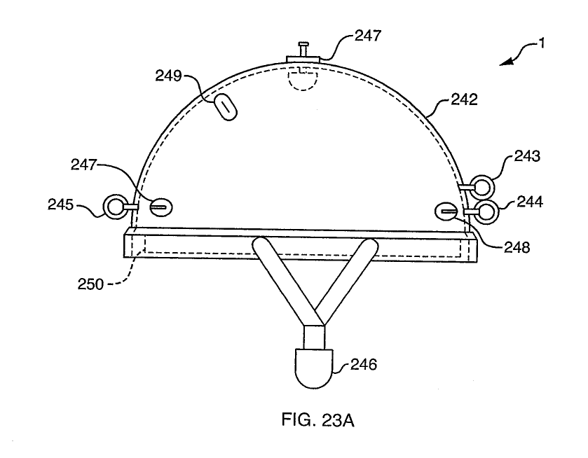

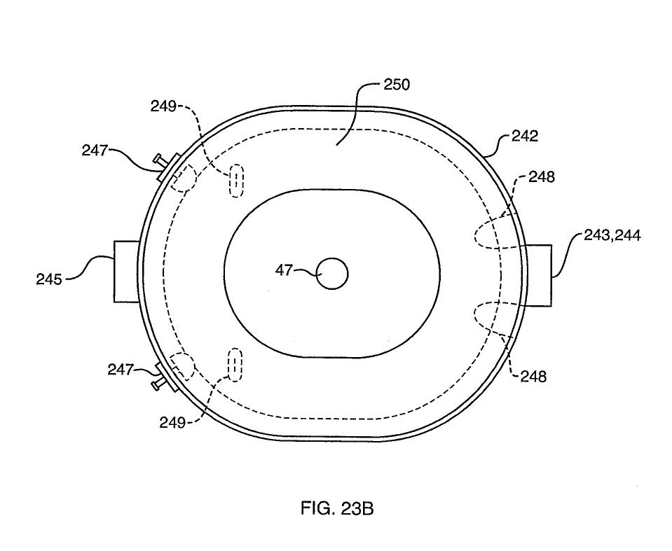

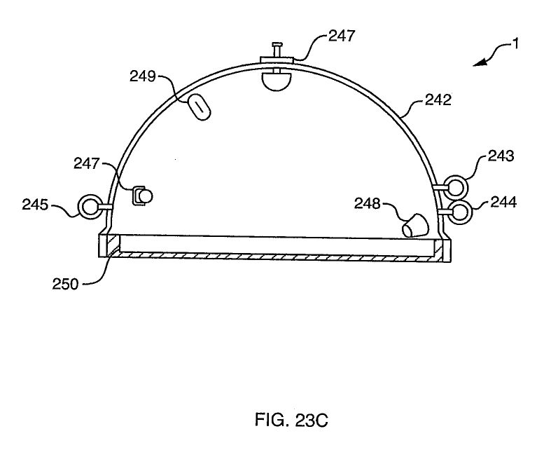

The cooling cap 1 includes a drain port 204, located at the back of the cap 1 (e.g., base of skull region) for drainage of the fluid from the fluid circulation space 197 after treatment of a patient. The drain port 204 can include a drain switch valve, illustrated in FIG. 22, activated by the presence or absence of fluid flow through the fluid circulation space 197.

FIG. 20 depicts in profile view the cooling cap mounted on a patient's head. The patient is lying on his back with the back of the cap down, and the front of the cap up. Dome 1 is a head covering structure. Seal band 205 is the sealing structure. Seal band 205 is depicted as a simple elastic band, and is the preferred embodiment for a cap with a flexible head covering structure. Water (fluid) inlet port 203 is depicted as being disposed at the back of the cap, but can be located anywhere on the cap, and can comprise more than one port into the circulation space. Vent port 202 is depicted at the back of the cap and is the preferred embodiment, but can be disposed in another location, and can comprise more than one port into the circulation space. Vent port 202 in one arrangement includes a pressure relief valve (not shown). Bottom drain port 204 is depicted at the back of the cap and is used to drain water from the cap for removal of the cap from the patient's head, and can comprise more than one port into the circulation space. Top drain port 206 is depicted at the front of the cap and is used to drain fluid (air and water) from the cap during the cooling cycle, and can include more than one port into the circulation space. Air bubbles 207 enter the cap through the band seal 205 and/or through vent port 202. Console connection elements (not shown) are disposed on the two drain ports, and the inlet port.

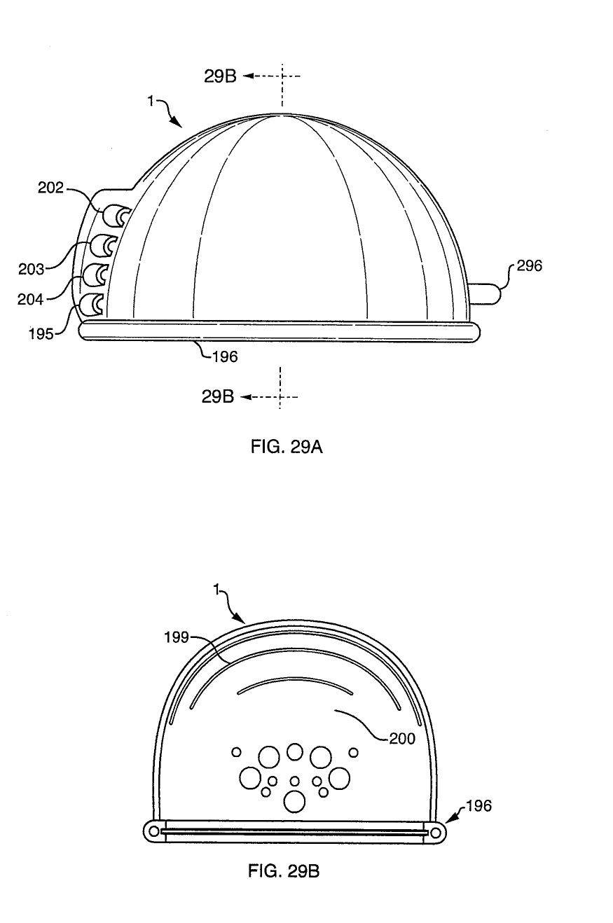

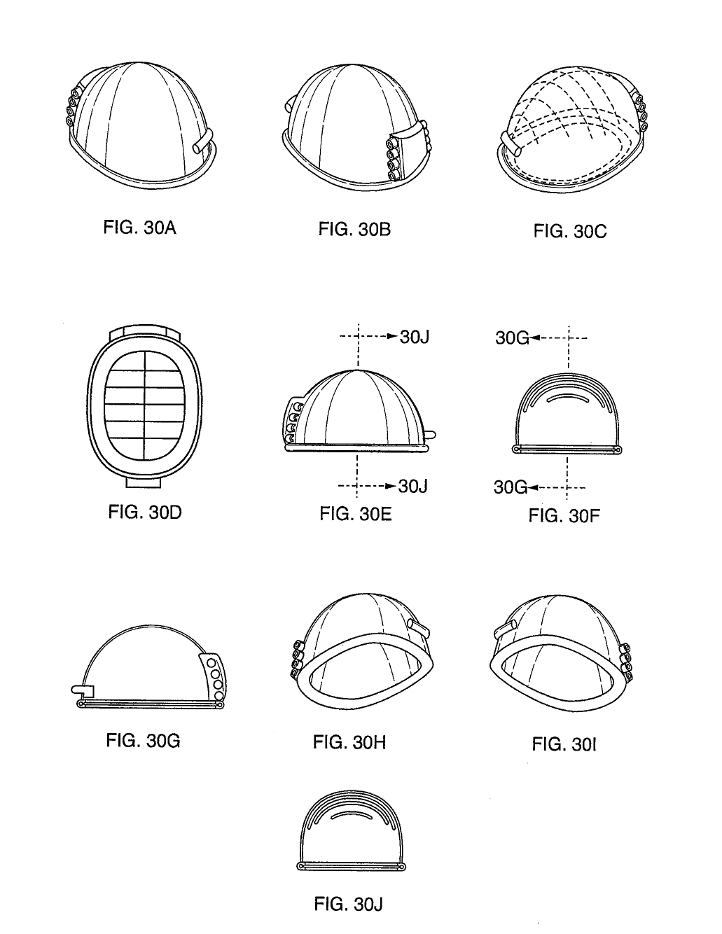

FIGS. 29A, 29B and 30 illustrate an arrangement of the cap 1. In one arrangement, the perimeter of the cap 1 includes a channel 196 (e.g., configured as a U-shaped channel or configured as a cylindrically shaped channel with multiple “holes” or openings) coupled to a vacuum source via a port 195. In the event fluid flows past the sealing element, the channel 196 scavenges the fluid and sends the scavenged fluid to the vacuum source. The channel 196 and vacuum source minimize leakage of fluid past the perimeter of the cap 1.

In one arrangement, the vacuum source that couples to the port 195 is distinct from the first pump 198 coupled to the fluid inlet 203 and is distinct from the second pump 199 coupled to the fluid outlet 206. As such, the vacuum source (e.g., third pump) controls the suction within the channel 196 independent relative to the operation of the first pump 198 or the second pump 199. As indicated above, operation of the vacuum source, in conjunction with the channel 196, scavenges fluid that flows past the sealing element. Additionally, operation of the vacuum source adjusts a pressure within the fluid circulation space 197 to maintain a slightly negative pressure within the fluid circulation space 197, thereby enhancing fluid flow from the fluid inlet 203 to the fluid outlet 206. The vacuum source also generates a negative pressure within the channel 196 to secure the cap 1 (e.g., the perimeter of the cap 1) to the patient's head.

In one arrangement, the channel 196 defines a continuous tube about the perimeter of the cap 1 and couples to a positive pressure source via the port 195. During operation, the positive pressure source delivers fluid at a positive pressure to the channel 196 to inflate the channel. Inflation of the channel 196 secures the cap 1 to the head of a patient and minimizes leakage of fluid within the fluid circulation space beyond the perimeter of the cap 1.

In one arrangement, the cap defines reinforcing structures or ribs 199. For example, in the case where the cap 1 is formed from a substantially compliant material such as a silicon material, the ridges provide structural stability to the cap 1. The ribs 199 limit over inflation of the cap 1 and minimized collapse of the cap 1 against the scalp of a patient. In one arrangement, the cap 1 includes a head support 200. The head support maintains a space between the head of the patient and the rear of the cap 1 such that the head of the patient does not bock the fluid inlet 203, vent port 202, or drain ports 204 associated with the cap 1.

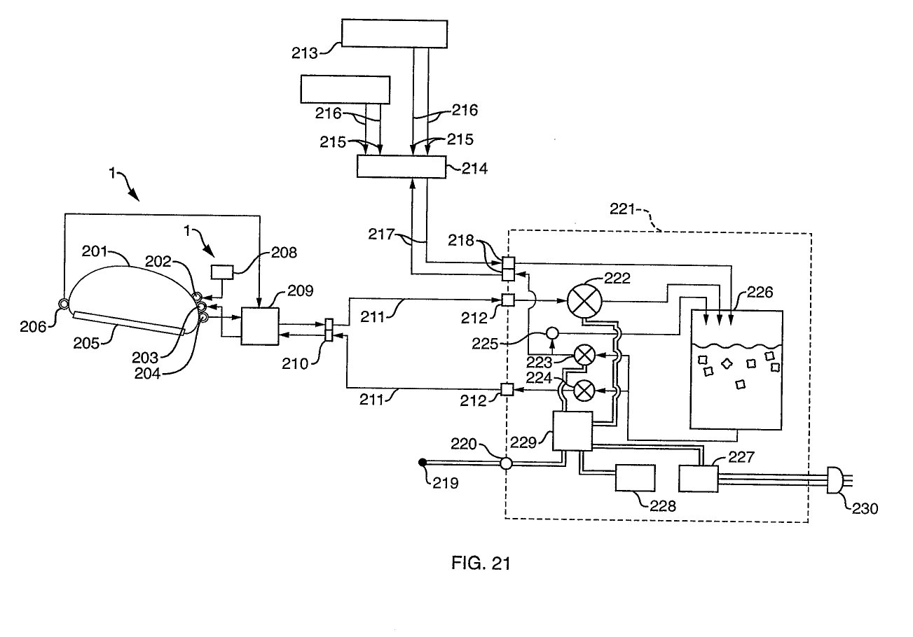

FIG. 21 is a simplified schematic of the system. The system comprises console 221, head cooling cap 201-210, umbilical 211, optional skin contact heat exchange pads and tubing set 213-218, and sensor 219. During the cooling cycle pump 224 draws cold water from reservoir 226 and delivers it to cooling cap circulation space through inlet port 203 at a modest positive pressure, and pump 222 draws fluid (air and water) from cooling cap circulation space through drain port 206 at a predetermined negative pressure and returns it to reservoir 226. Drain switch valve 209 responds to the positive pressure from pump 224 and connects drain port 206 to inlet of pump 222. When pump 224 is stopped, drain switch valve 209 disconnects drain port 206 from inlet of pump 222 and connects drain port 204 to inlet of drain pump 222 thereby draining the cap 1 of water. Pressure relief valve 208 maintains a predetermined negative pressure within the circulation space of the cooling cap. Control circuits control the operation of pumps 222, 223, and 224 according to signals received by body sensor 219.

FIG. 22 depicts a cross sectional view of switch 209. Switch 209 comprises a housing with a cylinder 232, a shuttle valve piston 233, a spring 234, fluid passage 235, cap bottom drain port 236, cap top drain port 237, and common drain port 238. The switch 209 operates as follows: 1) When there is no water flow, and therefore no pressure in fluid passage 235 spring 234 biases shuttle valve piston into position shown and fluidly connects bottom drain port 236 to common drain port 238. When the cooling cycle is activated, pressure in fluid passageway 235 moves shuttle valve piston to a second position and fluidly connects top drain port 237 to common drain port 238. The first position allows the cap 1 to be drained of water, and the second position fills the cap 1 with water.

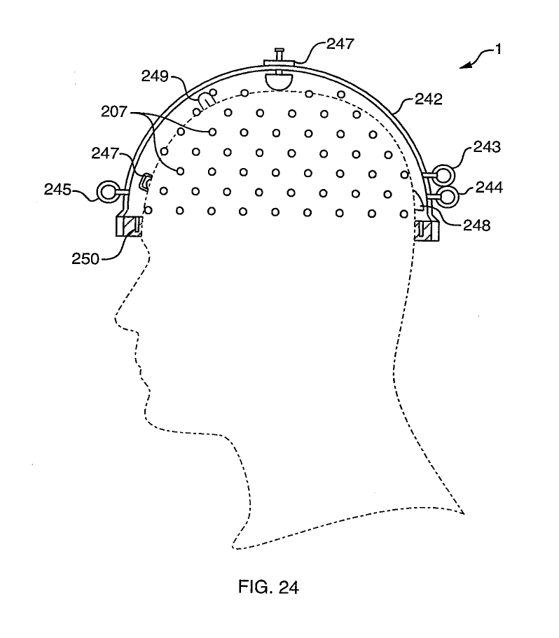

FIG. 23A depicts a rigid embodiment of the cooling cap 1. In this embodiment, all air enters the circulation space through the sealing structure. In this embodiment there is at least one intracranial probe port 249, and height adjustable standoffs 247. FIG. 23B depicts the sealing structure 250 that includes an elastic membrane as depicted. See FIG. 24 for function of this seal embodiment. FIG. 23C depicts a cross sectional view of the rigid cooling cap.

FIG. 24 depicts the rigid embodiment of the cooling cap mounted on a patient's head.

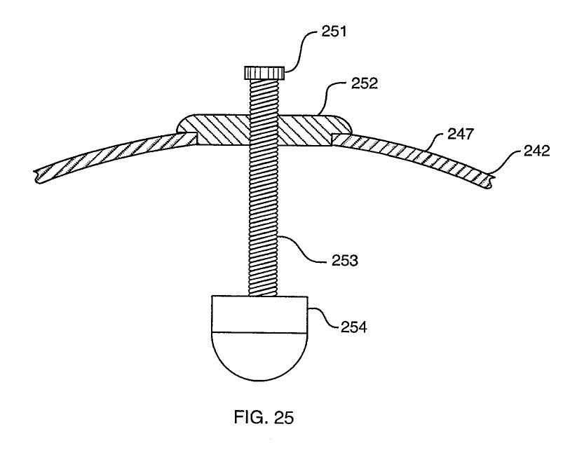

FIG. 25 depicts in cross sectional view the height adjustable standoff 47.

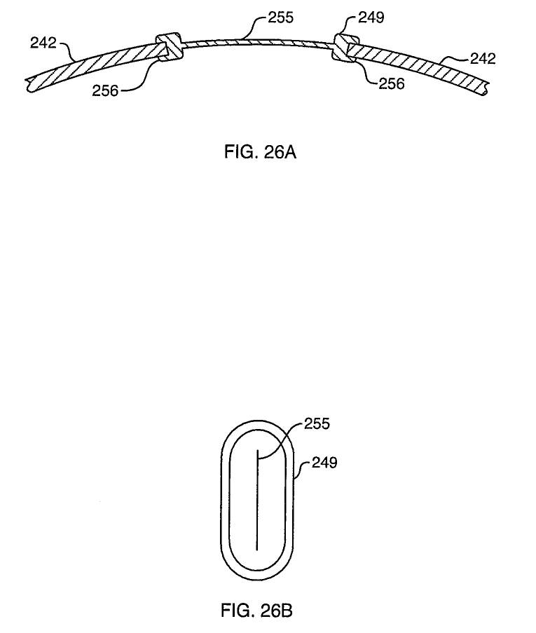

FIG. 26A depicts in cross sectional view the intracranial probe port 249 (e.g., a craniotomy device insertion location to seal a craniotomy device to the cap 1 and minimize leakage of fluid past the insertion location) mounted in the wall of the cooling cap 1. The port is molded from an elastic material as shown and is bonded into the wall of the cooling cap. FIG. 26B depicts the port as being a slit 55. Many other configurations of the port are possible.

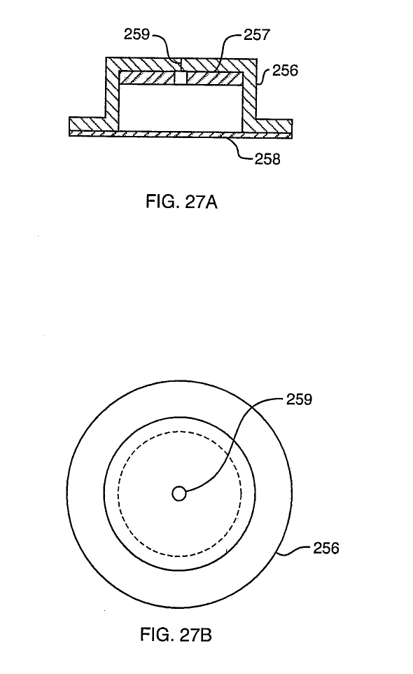

FIG. 27A depicts in cross sectional view the craniotomy protection device (e.g., a craniotomy seal cup that attaches to the patient's skull and minimizes leakage of fluid from the fluid circulation space into the associated craniotomy location). FIG. 27B depicts a top view of the craniotomy protection device. The craniotomy protection device keeps the craniotomy incision dry during head cooling. This is a practical requirement for using a direct water contact type of cooling cap with an intracranial probe.

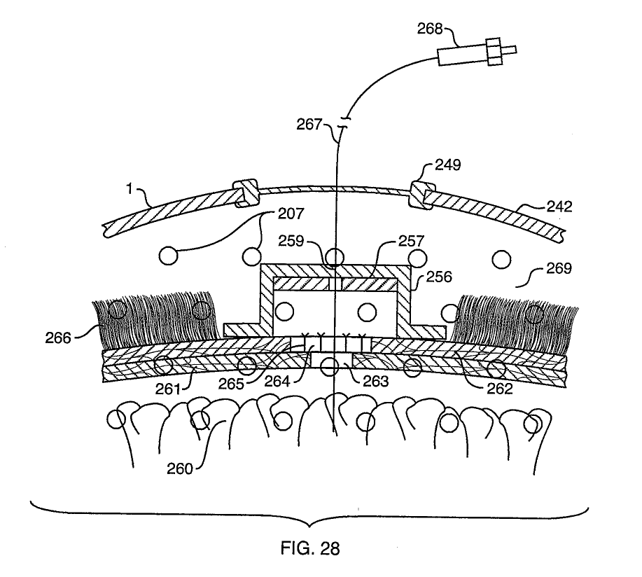

FIG. 28 depicts in cross section the craniotomy device, and intracranial probe and the cooling cap mounted on a patient's head.

While this invention has been particularly shown and described with references to preferred embodiments thereof, it will be understood by those skilled in the art that various changes in form and details may be made therein without departing from the spirit and scope of the invention as defined by the appended claims.

As described above, in one arrangement each of the collar 20, axilla pad 22, and back pad 24 couple to a console 2 as illustrated in FIG. 3. During operation, the user or clinician controls three variables associated with the console 2: an ON/OFF power supply 23, an amount of ice contained in a reservoir 25, and a number of accessories attached to the system, including a throttling valve position 27. The need to replace ice in the reservoir 25 is indicated by a temperature reading on a display (e.g., an LCD) on the pump cart or console 2. Such description is by way of example only. In one arrangement, the reservoir 25 includes a refrigeration unit that adjusts the temperature of the fluid contained in the reservoir 25. The refrigeration unit limits the need for a user to replace ice within the reservoir 25 to maintain fluid within the reservoir 25 at a particular temperature or within a given temperature range.

As illustrated in FIG. 1, the thermal regulation system 15 includes the head covering device 1, the console 2, the body covering device 6, and the body temperature sensor 10. In one arrangement, embodiments of the thermal regulation system 15 form part of a resuscitation system that includes defibrillation, chest compression, fluid infusion, or other mechanisms necessary to or used in the resuscitation process.

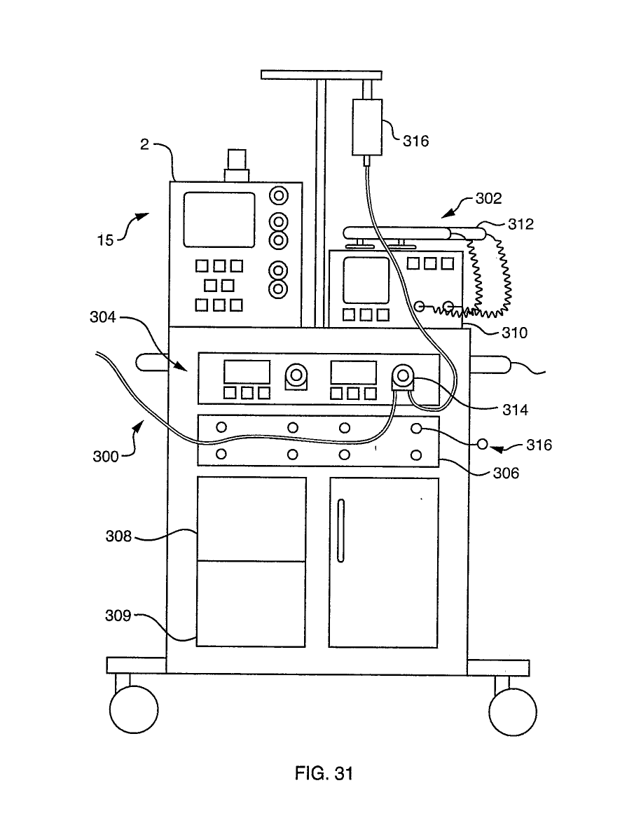

FIG. 31 illustrates an example resuscitation system 300. The resuscitation system 300 includes a thermal regulation system 15 (e.g., the console 2 of which is shown), a defibrillation apparatus 302, a fluid treatment apparatus 304, a physiologic monitoring apparatus 306, a ventilator 308, and a chest compression apparatus 309.

The defibrillation apparatus 302 includes a defibrillator 310 and defibrillator electrodes 312. When applying the defibrillation electrodes 312 to a patient and activating the defibrillator 310, a user applies an electrical current to the patient's heart to restore a normal rhythm to the patient's heart.

The fluid treatment apparatus 304, in one arrangement, includes a fluid infusion pump 314 that provides metered infusion of fluids into the patient. The pump 314 delivers the fluids from a fluid bag 316, such as a Ringer's solution, to the patient to maintain a hydration level of the patient. In another arrangement the pump delivers a fluid medicament from the fluid bag 316 to the patient to aid in patient resuscitation.

The physiological monitor 306 detects a physiologic state of a patient. For example, the physiological monitor 180 is configured as an electrocardiogram (EKG) sensor, a heart monitoring sensor, a temperature sensor, or a pulse oximetry sensor. The resuscitation system 300 can adjust delivery of thermal exchange fluid from the thermal regulation system 15, to adjust or maintain the patient's body temperature of a patient, based upon the signals received from an associated physiological monitor 316.

The ventilator 308 couples to a patient airway and provides oxygen and other gasses to the patient, thereby providing inhalation therapy to the patient and aiding in the resuscitation of the patient. The chest compression apparatus 309 couples to the chest of a patient to cyclically compress the patient's chest and aid in the resuscitation of the patient.

1. A method of cooling a head of a patient to induce hypothermia, comprising:
covering at least a portion of the head of the patient with a head cooling device, the head cooling device forming a fluid circulation space bounded by a surface of the head cooling device and a surface of the patient's head when the device is worn on the patient's head;
circulating cooling liquid through the fluid circulation space while the fluid circulation space is held at a negative gage pressure; and
introducing a gas into the fluid circulation space to induce turbulence within the cooling liquid.

2. The method of claim 1, wherein the step of circulating cooling liquid further comprises introducing the cooling liquid into the fluid circulation space at a lower level than where the cooling liquid leaves the fluid circulation space.

3. The method of claim 1, wherein the step of introducing a gas further comprises creating bubbles in the cooling liquid to induce turbulence.

4. The method of claim 1, wherein the step of introducing a gas further comprises allowing air to enter the fluid circulation space by passing air under a head-contacting sealing structure of the head cooling device.

5. A system for head cooling comprising:
a head cooling device configured to define a fluid circulation space bounded by a surface of the head cooling device and a surface of the patient's head when the device is worn on the patient's head, the fluid circulation space in fluid communication with a liquid inlet port located at the back of the device and a liquid outlet port located at the front of the device, the head cooling device including a sealing structure configured to contact the patient's head and to maintain a negative gage pressure between a turbulent, flowing gas/liquid mixture within the fluid circulation space and ambient atmosphere outside the head cooling device; and
at least one pump mechanism in fluid communication with the fluid circulation space for establishing negative gage pressure within the fluid circulation space.

6. The system of claim 5, wherein the head cooling device further comprises a gas inlet port for delivering gas into the fluid circulation space.

7. The system of claim 5, wherein the at least one pump mechanism comprises:
a first pump mechanism in fluid communication with the liquid inlet port for delivering fluid into the fluid circulation space; and
a second pump mechanism in fluid communication with the liquid outlet port for removing fluid from the fluid circulation space, the second pump mechanism configured to create a higher volumetric flow rate than the first pump mechanism.

8. The system of claim 5, wherein the sealing structure is configured to allow air to enter the fluid circulation space while hindering liquid from leaking past the sealing structure.

9. The system of claim 5, wherein the negative gage pressure is between about −0.2 to about −2.0 PSIG.

10-71. (canceled)
